(19) United States
(12) Patent Application Publication
(10) Pub. No.: US 2006/0105367 A1
(43) Pub. Date: May 18, 2006

(54) Method and markers for the diagnosis of Graft versus Host Disease (GvHD)

(76) Inventor: Harald Mischak, Sehnde (DE)

Correspondence Address: JACOBSON HOLMAN PLLC, 400 SEVENTH STREET N.W., SUITE 600, WASHINGTON, DC, 20004, US

(21) Appl. No.: 11/249,737

(22) Filed: Oct. 14, 2005

Related U.S. Application Data
(60) Provisional application No. 60/618,177, filed on Oct. 14, 2004.

(51) Int. Cl. C12Q 1/68; G01N 33/537; G01N 33/00

(52) U.S. Cl. 435/6; 435/7.92; 436/86

(57) ABSTRACT

A method for the diagnosis of GvHD, the method comprising: c) measuring the presence or the absence of a polypeptide marker in a urine sample, wherein the polypeptide marker is selected from the group of polypeptide markers shown in table 1, and d) comparing the probability of the presence of this marker in a disease patient to the probability of the presence of this marker in a control patient, wherein c1) if the probability of the presence of this marker in a disease patient is higher than the probability of the presence of this marker in a control patient, the presence of this marker is indicative for a higher probability of having the disease rather than the control condition, or c2) if the probability of the presence of this marker in a disease patient is lower than the probability of the presence of this marker in a control patient, the absence of the marker is indicative for a higher probability of having the disease rather than the control condition.

This is a complete application claiming benefit of U.S. provisional No. 60/618,177, filed on Oct. 14, 2004.

The present invention relates to the diagnosis of Graft versus Host Disease (GvHD).

GvHD is a complication of stem cell transplantation, one of the most effective therapy against blood cancer. The major forms of blood cancer are lymphoma, leukemia and multiple myeloma. These cancers are formed either in the bone marrow or the lymphatic tissues of the body. They affect the way your body makes blood and provides immunity from other diseases. Overall survival rates for people with blood cancer have doubled in the past 30 years because of more effective radiation and chemotherapy treatments. In 1960, only 4 percent of children diagnosed with childhood leukemia survived. Today, 79 percent are expected to live if they receive the best treatment available. Still, leukemia is the leading cause of death by disease in children. Adults are more likely than children to get blood cancer, since the risk increases with age. In 2003, it was estimated that about 106,200 Americans will be diagnosed with one of the blood cancers and about 57,500 will die from the disease. Lymphomas account for approximately 55 percent of new cases, leukemia about 28 percent, and myeloma about 14 percent Less common forms of blood cancers account for about 3 percent of cases.

Allogeneic hematopoietic peripheral blood stem cell transplantation (allo-HSCT) is applied with success to many hematopoietic malignancies. Despite its curative potential, the application of allo-HSCT is limited by life-threatening complications such as severe acute graft-versus-host disease (GvHD). Depending on the type of transplantation, the immunosuppressive treatment and the underlying disease, between 35% and 70% of patients develop GvHD, requiring immunosuppressive treatment in more than 35% of patients. Early diagnosis and better control of GvHD will be necessary to increase the safety of allo-HSCT, also with respect to a broader application of HSCT.

Currently, diagnosis of GvHD is mainly based on clinical parameters such as skin rash, diarrhea, elevation of serum liver enzymes or other. Differential diagnosis of GvHD depends on organ biopsies to distinguish GvHD from other common complications that present with similar clinical symptoms e.g. sepsis or reactivation of endogenous viruses and medication-induced side effects. The use of biomarkers such as differentially expressed or excreted polypeptides and proteins has the potential of improving early and accurate diagnosis of GvHD and other complications of allo-HSCT without requiring invasive procedures, such as biopsies. Single molecules are currently described as potential markers for GvHD but data of all molecules potentially involved are not yet reported. Due to a lack of suitable technology, the search for polypeptides or proteins involved in GvHD up to now has been naturally biased by the preferential analysis of known molecules with potential patho-physiological importance.

Consequently, there is a need for a fast and simple method and means for diagnosis of GvHD and for the differential diagnosis to distinguish GvHD from other complications like sepsis.

Accordingly, the object of the present invention is to provide methods and means for the diagnosis and differential diagnosis of GvHD.

An analytic display of all proteins and peptides present or changed after allo-HSCT might allow to gain significantly more insight in the development of and processes involved in GvHD. Proteomics is being developed to characterize and identify the molecules significant for different diseases enabling early identification of biomarkers and early intervention in cancer. A technique, allowing the reproducible analysis of all polypeptides present in complex biological samples within short time and suitable for high throughput analysis was recently developed by our group (Wittke S. et al., 2003, Proteomics for clinical diagnostic and establishment of new markers and therapeutic targets: determination of proteins and peptides in urine with CE-ESI-TOF-MS. Journal of Chromatography A, 1013: 173-181). The stable on-line coupling of capillary electrophoresis and mass spectrometry (CE-MS), together with advances in software analysis, led to the display of >1000 polypeptides present in individual samples, identified via their particular migration time in the CE and their actual mass. All data generated from individual samples are stored in a data base, allowing intraindividual comparison of the samples taken at different time points, yielding a patient-specific pattern, as well as comparison of patient groups and controls. Screening of urine of healthy volunteers led to the establishment of a “normal urine polypeptide pattern”, consisting of more than 500 polypeptides. This allowed the comparison to patterns obtained from patients with different diseases and led to the detection of polypeptide-patterns indicative for the health status of individuals.

Using this method, we were able to find markers for GvHD, as shown in Table 1. The problem of diagnosis of GvHD is solved by the use of at least one polypeptide marker in a urine sample for the diagnosis of GvHD, wherein the polypeptide marker is selected from the group of markers shown in table 1. The problem of differential diagnosis to distinguish GvHD from sepsis is solved by the use of at least one polypeptide marker in a urine sample for the differential diagnosis of GvHD, wherein the polypeptide marker is selected from the group of markers shown in table 2.

The present invention has numerous advantages compared to the state of the art. First, the presence of polypeptide markers according to the invention can be determined in urine samples. Therefore, there is no need to take biopsies. Thus, the present invention allows a simplified and fast diagnosis of GvHD, allowing to screen patients regularly for the presence of GvHD and to diagnose GvHD at early stage. Furthermore, the polypeptide markers according to the invention can be used for differential diagnosis between GvHD and other complications of HSCT like sepsis. The high number of markers identified according to the present invention allows to increase both specificity and sensitivity of diagnosis as compared to the use of only a single or a small number of markers. Also, the present invention provides methods which allow to measure said polypeptide markers without the use of specific ligands such as antibodies or aptamers.

The polypeptide markers as shown in the tables have been identified by capillary electrophoresis-mass spectrometry (CE-MS). Starting from the parameters defining the polypeptide markers, it is possible by methods known in the art to identify the sequence of the corresponding polypeptides and then to synthesize or produce the corresponding polypeptides, e.g. with the help of protein synthesis or expression of the corresponding gene in appropriate cells.

The markers are defined by there mass and their migration time in capillary electrophoresis (CE), particularly mass and their migration time obtained according to Example 1. It is known that CE migration times can vary, typically in the range of 5 min, more typically in the range of 3 minutes. However, the sequence of markers being eluted is typically the same or very similar for each CE system applied. The system can be calibrated by use of polypeptides which are present in almost any urine sample, e.g. by the polypeptides given in tables 3.

Variation of the masses between measurements or between different mass spectrometers is relatively small; typically it is in the range of plus or minus 0.05%.

In table 1, polypeptide markers are listed which are preferred for the discrimination between healthy individuals and individuals suffering from GvHD. Polypeptides 1 to 7 have higher frequency in the GvHD group than in control, polypeptides 9-16 have lower frequency in GvHD group than in control. Polypeptide 8 has higher mean amplitude in GvHD group than in control

In table 2, polypeptide markers are listed, which can be used for a differential diagnosis of GvHD and sepsis. Polypeptides 17 to 25 have lower frequency in the GvHD group than in sepsis group; polypeptides 26-29 have higher frequency in GvHD group than in sepsis.

In table 3, polypeptides are listed, which are preferred as internal standards to standardize the CE-time.

In table 4, clinical data of HSCT patients are listed whose samples were used for identification of polypeptide markers according to Example 1. Patients with no complications during the observation period are marked with ‘N’. Patients with “Fever” had bacteria in the blood culture in combination with fever on the indicated days and were treated with specific antibiotics. Patients with GvHD are marked “Y” and the date of the diagnosis is given in the column “after HSCT”. No additional complications were reported in patients with GvHD, unless “other complications” shows an entry of the particular problems/complications. Abbreviations: HSCT: hematopoietic stem cell transplantation; MUD PBSCT: matched unrelated donor peripheral stem cells; SIB-PBSC: HLA-identical sibling donor peripheral blood stem cell transplantation; MM: mismatched unrelated donor; h PBSC/BMT: haploidentical peripheral blood and bone marrow transplantation; auto: autologous stem cell transplantation; RIT: radio-immunotherapy; TBI: total body irradiation; n.a.: not applicable; N: no GvHD; Y: GvHD; FUO: fever of unknown origin

The polypeptide markers used according to the present invention can be identified and their presence can be measured in urine samples. Urine samples can be taken as known in the state of the art. Preferably, midstream urine is used in the context of the present invention.

The polypeptide markers used according to the present invention can be gene expression products such as proteins, peptides, and fragments or other degradation products of proteins or peptides. They can be modified by posttranslational modifications, e.g. by glycosylation, phoshorylation, alkylation or disulfide bond. It is known that fragments and degradation products can have a different diagnostic value and/or physiological role than the protein or peptide they have been derived from. For example, in different diseases, different proteolytic degradation products or fragments can be found. It is also considered to be within the scope of the present invention if the urine sample is pretreated to chemically modify the polypeptide markers contained in the urine and to measure these chemically modified polypeptide markers. The polypeptide markers according to the present invention have a molecular mass between 400 and 20,000 Da, particularly between 700 and 14,000 Da, more particularly between 800 and 11,000 Da.

In the context of the present invention, diagnosing or diagnosis means that, for an individual patient, the probability of having the respective disease is determined.

Diagnosis may also include confirming a preliminary diagnosis, particularly a preliminary diagnosis established by a different method.

Furthermore, in a preferred embodiment, diagnosis according to the present invention particularly relates to “differential diagnosis”. The term “differential diagnosis” relates to distinguishing between two different diseases, i.e. to determining for an individual patient the probability of having a certain first disease as compared to having a certain second disease. More particularly, differential diagnosis according to the present invention relates to distinguishing between GvHD and sepsis.

In another embodiment, the present invention relates to a method for the diagnosis of GvHD and the differential diagnosis between GvHD and Sepsis, the method comprising:
a) measuring the presence or the absence of a polypeptide marker in a urine sample, wherein the polypeptide marker is selected from the group of polypeptide markers shown in table 1 to 2, and
b) comparing the probability of the presence of this marker in a disease patient to the probability of the presence of this marker in a control patient, wherein
c1) if the probability of the presence of this marker in a disease patient is higher than the probability of the presence of this marker in a control patient, the presence of this marker is indicative for a higher probability of having the disease rather than the control condition (e.g. Marker No. 1 (mass 1965.80), probability of presence in a disease patient is 82%, probability of presence in a control patient is 18%), or
c2) if the probability of the presence of this marker in a disease patient is lower than the probability of the presence of this marker in a control patient, the absence of the marker is indicative for a higher probability of having the disease rather than the control condition (e.g. Marker No. 16 (mass 1848.10), probability of presence in a disease patient is 9%, probability of presence in a control patient is 82%).

Preferably, the individual probabilities according to step b) are as indicated in the tables 1 and 2 (“frequency”).

The term “measuring” according to the present invention relates to determining the presence of a polypeptide or other substance of interest.

The decision whether a polypeptide marker is present or absent may depend on definition of a suitable threshold value. The threshold value can either be defined through the sensitivity of the method of measurement, or it can be defined at will. The threshold in the context of the present invention is 25 fmol/μl in a sample which has been injected into a mass spectrometer according to Example 1. However, this threshold may be the same when other methods are used. This threshold coincides with the detection threshold of a typical mass spectrometer. This threshold corresponds approximately to a concentration of the polypeptide marker in the urine sample of 50-5000 pmol/l. If different thresholds are to be used (e.g. when using another detection method), the corresponding probabilities may differ, but can easily be established by the person skilled in the art.

Tables 1 to 2 list the probability (also designated as “frequency” in the tables) of a given polypeptide marker being present in a urine sample of a healthy control patient or a control patient suffering from a certain disease. The discrimination factor indicates the difference between the probability of presence in the disease as compared to a given control condition. The discrimination factor can easily be calculated from the respective probabilities. The higher the discrimination factor, the better is the potential of the given marker to distinguish between the disease and the control condition. An absolute value of the discrimination factor of 0.40 or higher is preferred. An example is given to explain the discrimination factor: Polypeptide 1 in table 1 (mass 1965.80) has a frequency of 82% in the GvHD group, and a frequency of 18% in the control group. The difference between these frequencies is 64%; this is an absolute value of 0.64.

The person skilled in the art is able to establish similar tables for the polypeptide markers by himself and/or to refine the data contained in the tables, e.g. based on further patient data and/or according to different thresholds for the presence of the polypeptide marker.

For diagnosis, the probability of the presence of the polypeptide marker in a disease patient is compared to the probability of the presence of this marker in a control patient, wherein the individual probabilities are as indicated in the tables. If the probability of the presence of this marker in a disease patient is higher than the probability of the presence of this marker in a control patient, then the presence of this marker in the sample is indicative that the patient from whom the sample originates has a higher probability of having the disease rather than the control condition. If the probability of the presence of this marker in a disease patient is lower than the probability of the presence of this marker in a control patient, then the absence of this marker in the sample is indicative that the patient from whom the sample originates has a higher probability of having the disease rather than the control condition.

Thus, diagnosis can be established according to statistical methods familiar to the person skilled in the art.

The invention can be carried out using only one of the polypeptide markers, e.g. polypeptide marker No. 1, 2, 3, 4, 5, or 16, 15, 14, 13, 12 for diagnosis of GvHD or marker No. 17, 18, 19, 20, 21 or 29, 28, 17, 26 for differential diagnosis between GvHD and Sepsis, or using a plurality of the polypeptide markers. Preferably, presence of a plurality of polypeptide markers is measured. Preferably at least 3 of the markers, e.g. for the diagnosis of GvHD from table 1:

Polypeptide marker No. 1, 2 and 3; 2, 3 and 4; 3, 4 and 5; or 16, 15 and 14; 15, 14 and 13; 14, 13 and 12; or 1, 2 and 16; 2, 3 and 16; 3, 4 and 16; 1, 2 and 15; 2, 3 and 15; 3, 4 and 15; 1, 2 and 14, 2, 3 and 14, 3, 4, and 14.

For the differential diagnosis between GvHD and Sepsis from table 2:

Polypeptide marker No. 17, 18 and 19; 18, 19 and 20; 19, 20 and 21; 20, 21 and 22; or 29, 28 and 27; 28, 27 and 26; or 17, 18 and 29, 18, 19 and 29; 19, 20 and 29; or 17, 18 and 28; 18, 19 and 28; 19, 20 and 28.

More preferably at least 10 of the markers, e.g. for the diagnosis of GvHD from table 1:

Polypeptide marker No. 1-10; 7-16; or 1-5 and 12-16.

For the differential diagnosis between GvHD and sepsis from table 2:

Polypeptide marker No. 17-26; 20-29; or 17-21 and 25-29

Most preferred all of the markers according to the present invention are measured.

An advantage of the present invention is that it provides a multitude of suitable markers. Measuring a plurality of markers can increase both sensitivity and selectivity of diagnosis. Therefore, also markers which show low discrimination factors between the disease and control can be used for diagnosis if they are combined with other markers.

If a plurality of polypeptide markers is used, a “pattern” is be generated which contains the information about the presence for each marker measured. This pattern can then be compared to the pattern of probabilities of presence of the polypeptide markers in a disease or control patient. Each table represents a pattern of probabilities of finding given polypeptide markers in certain disease and control patients.

Therefore, in a preferred embodiment, the present invention relates to a method for the diagnosis of GvHD and for the differential diagnosis between GvHD and sepsis, the method comprising:
a) establishing a pattern of presence or absence for a plurality of polypeptide markers in a urine sample, wherein at least two polypeptide markers are selected from the group of polypeptide markers shown in table 1 to 2, e.g. marker No. 1 to 16 in combination with any of the other markers selected from table 1 for the diagnosis of GvHD or marker No. 17 to 29 in combination with any of the other markers selected from table 2 for the differential diagnosis between GvHD and Sepsis, and
b) comparing the probability of finding this pattern in a disease patient to the probability of finding this pattern in a control patient, wherein
c1) if the probability of finding the pattern in a disease patient is higher than the probability of the finding the pattern in a control patient, finding this pattern is indicative for a higher probability of having the disease rather than the control condition, or
c2) if the probability of finding the pattern in a disease patient is lower than the probability of the finding the pattern in a control patient, finding this pattern is indicative for a lower probability of having the disease rather than the control condition, or

Preferably, the individual probability for the at least two polypeptide markers (e.g. marker No. 1 to 16 in combination with any of the other markers selected from table 1 for the diagnosis of GvHD or marker No. 17 to 29 in combination with any of the other markers selected from table 2 for the differential diagnosis between GvHD and Sepsis) according to step b) is as indicated in the tables 1 and 2.

Comparison of the found pattern with the probability of finding the pattern in a disease or control patient can be performed according to statistical methods known in the art. Preferably, automated methods are employed, e.g. CART-analysis, random forest analysis, and support vector machines (SVM, see e.g. Xiong. M., et al. (2001). Biomarker identification by feature wrappers. Genome Research vol. 11, p. 1878-1887). Comparison can also be performed simultaneously for several different patterns and the probability of finding them.

Thus, the measured pattern is typically compared to the probability of finding the pattern in at least two different conditions.

If necessary, the urine samples may be pre-treated before measurement of the polypeptide marker. Particularly, lipids, nucleic acids or polypeptides may be purified from the sample according to methods known in the art, including filtration, centrifugation, or extraction methods such as chloroform/phenol extraction.

Measuring the presence of a polypeptide marker can be done by any method known in the art.

Preferred methods include gas phase ion spectrometry, such as laser desorption/ionization mass spectrometry, surface enhanced laser desorption/ionization time-of flight mass spectrometry (SELDI-TOF MS) and CE-MS. These spectrometry methods allow measuring the polypeptide markers without the need for ligands such as antibodies or aptamers.

Urine sample generally are highly complex, i.e. they contain numerous polypeptides. In case of high complexity, a spectrometric analysis becomes difficult. To reduce the complexity of the sample, the polypeptides contained in the sample may be separated by any suitable means, e.g. by electrophoretic separation, affinity-based separation, or separation based on ion exchange chromatography. Particular examples include gel electrophoresis, two-dimensional polyacrylamide gel electrophoresis (2D-PAGE), capillary electrophoresis, metal-affinity chromatography, immobilized metal-affinity chromatography (IMAC), affinity chromatography based on lectins, liquid chromatography, high pressure liquid chromatography (HPLC), and reversed-phase HPLC, cation exchange chromatography, and selectively binding surfaces (such as the surfaces used in SELDI-TOF).

However, the most preferred method is CE-MS, in which capillary electrophoresis (CE) is coupled to mass spectrometry (MS). CE-MS has been described in detail elsewhere (see e.g. German patent application DE 100 21 737, and Kaiser, T., et al., Capillary Electrophoresis coupled mass spectrometry to establish polypeptide patterns in dialysis fluids. J Chromatogr A, vol. 1013, p. 157-171(2003)).

CE is known to the person skilled in the art. In brief, the sample is loaded onto an electrophoresis capillary and a voltage of up to 50 kV, typically up to 30 kV, is applied. Typical capillaries are fused silica capillaries, i.e. glass capillaries comprising an outer sheath as mechanical support and to improve mechanical flexibility, e.g. a sheath made of thermoplastic material. Typically, the capillary is untreated, i.e. it shows hydroxy-groups on its inside. However, the capillary may also be coated on the inside. E.g., hydrophobic coating can be used to improve discriminatory power. In addition to the voltage, also pressure may be applied, which is typically in the range of 0 to 1 psi. The pressure can also be applied or increased during the run.

To improve discriminatory power, also a stacking protocol can be applied when loading the sample: Before loading of the sample, a base is loaded, then the sample is loaded, then an acid. The principle is to capture the analyte ions between a base and an acid. If voltage is applied, the positively charges analyte ions move towards the base. There, they get negatively charged and move into the opposite direction towards the acid, where they get positively charged. This stacking repeats itself until acid and base are neutralized. Then, the separation starts from a well concentrated sample.

The sample is contained in an appropriate buffer in which polypeptides are soluble, e.g. phosphate buffer. For CE-MS coupling, it is preferred to use volatile solvents and to work under mostly salt-free conditions to avoid contamination of the MS. Examples comprise acetonitrile, isopropanol, methanol, and the like. The solvents can also be combined with water and a weak acid (e.g. 0.1% formic acid), the latter to protonate the analyte. The polypeptides in the sample are separated according to size and charge, which determine the run-time in the capillary. CE is characterized by high separating power and short time of analysis.

For subsequent MS analysis, either fractions collected from the CE can be analyzed as separate batches or, preferably, the CE system can be coupled via a suitable interface to the mass spectrometer to allow continuous flow analysis. Alternatively, the flow from the CE may be used to generate continuous “separation tracks”, which can be analyzed separately.

In the mass spectrometer, ions generated from the sample are analyzed according to the mass/charge (m/z) quotient. Using mass spectrometry, it is possible to routinely analyze 10 fmol (i.e. 0.1 ng of a 10 kDa polypeptide) with a precision of ±0.01%. Experimentally, it is possible to analyze even less than 0.1 fmol.

Any type of mass spectrometer can be used. In mass spectrometers, an ion-generating device is coupled with an suitable analyzer. For example, the electrospray ionization (ESI) interfaces are most commonly used to produce ions from liquid samples, whereas MALDI is most commonly used to produce ions from individually processed samples. Different kinds of analyzers are available, e.g. ion trap analyzers or time-of-flight (TOF) analyzers. Both ESI and MALDI can be combined with essentially all types of mass spectrometers, although ESI has usually been combined with ion traps, whereas MALDI has usually been combined with TOF.

A preferred CE-MS method according to the present invention includes capillary electrophoresis coupled online via ESI to a TOF analyzer.

The CE-MS technique permits to measure the presence of several hundred polypeptide markers simultaneously in a short time in a small volume with high sensitivity. Once the presence of the polypeptide markers has been measured, a pattern of the measured polypeptide markers is generated and can be compared to a disease pattern by any of the methods described further above. However, in many cases it will be sufficient for diagnosis to measure only one or a limited number of the markers.

The polypeptide sequences can be determined according to methods well-known to the person skilled in the art (see e.g. C. S. Spahr et al. (2001). Towards defining the urinary proteome using liquid chromatography-tandem mass spectrometry. I. Profiling an unfractionated tryptic digest. Proteomics vol. 1, p. 93-107).

Depending on the type of polypeptide marker, it is possible to measure its presence or absence by further means. For example, if the polypeptide is biologically active, its presence may be determined by cellular or enzymatic assays.

Presence of a polypeptide can also be determined by use of ligands binding to the polypeptide of interest. Binding according to the present invention includes both covalent and non-covalent binding.

A ligand according to the present invention can be any peptide, polypeptide, nucleic acid, or other substance binding to the polypeptide of interest. It is well known that polypeptides, if obtained or purified from the human or animal body, can be modified, e.g. by glycosylation. A suitable ligand according to the present invention may bind the polypeptide also via such sites.

Preferred ligands include antibodies, nucleic acids, peptides or polypeptides, and aptamers, e.g. nucleic acid or peptide aptamers. For many polypeptides, suitable ligands are commercially available. Furthermore, methods to generate suitable ligands are well-known in the art. For example, identification and production of suitable antibodies or aptamers is also offered by commercial suppliers.

The term “antibody” as used herein includes both polyclonal and monoclonal antibodies, as well as fragments thereof, such as Fv, Fab and F(ab)₂ fragments that are capable of binding antigen or hapten.

Preferably, the ligand should bind specifically to the polypeptide to be measured. “Specific binding” according to the present invention means that the ligand should not bind substantially to (“cross-react” with) another polypeptide or substance present in the sample investigated. Preferably, the specifically bound protein or isoform should be bound with at least 3 times higher, more preferably at least 10 times higher and even more preferably at least 50 times higher affinity than any other relevant polypeptide.

Non-specific binding may be tolerable, particularly if the investigated peptide or polypeptide can still be distinguished and measured unequivocally, e.g. according to its size on a Western Blot, or by its relatively higher abundance in the sample.

A method for measuring the presence of a polypeptide of interest may comprise the steps of (a) contacting a polypeptide with a specifically binding ligand, (b) (optionally) removing non-bound ligand, (c) measuring the presence or amount of bound ligand.

Binding of the ligand can be measured by any method known in the art. Thus, suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting), can be used alone or in combination with labeling or other detection methods as described above.

The ligand may also be present on an array. Said array contains at least one additional ligand, which may be directed against a peptide, polypeptide or a nucleic acid of interest. Said additional ligand may also be directed against a peptide, polypeptide or a nucleic acid of no particular interest in the context of the present invention. Preferably, ligands for at least five, more preferably at least 10, even more preferably all polypeptide markers according to the present invention are contained on the array.

According to the present invention, the term “array” refers to a solid-phase or gel-like carrier upon which at least two compounds are attached or bound in one-, two- or three-dimensional arrangement. Such arrays (including “gene chips”, “protein chips”, antibody arrays and the like) are generally known to the person skilled in the art and typically generated on glass microscope slides, specially coated glass slides such as polycation-, nitrocellulose- or biotin-coated slides, cover slips, and membranes such as, for example, membranes based on nitrocellulose or nylon.

The array may include a bound ligand or at least two cells expressing each at least one ligand.

The invention is further illustrated by the following examples:

### EXAMPLE 1

Patients:

The protocol for this study was approved by the local ethic committees and informed consent was obtained from all participants. Forty patients transplanted at the Hannover Medical School, the University of Regensburg and the University of Munich were included in the analysis. 35 patients (26 with acute myeloid leukemia (AML), 4 with acute lymphocytic leukemia (ALL), 1 with high risk chronic myeloid leukemia (CML), 1 with non-Hodgkin lymphoma (NHL), 1 with follicular lymphoma, 1 with multiple myeloma (MM) and 1 with myelodysplastic syndrome (MDS)) were transplanted from allogeneic donors, while 5 (1 AML, 2 MM, 2 NHL) received autologous stem cells. Urine samples were collected prior to conditioning and then twice a week from each patient over 20 to 100 days after HSCT.

In addition, samples from 5 patients from the intensive care unit (ICU) with severe septic complications were included.

Conditioning and Transplantation:

Nineteen patients were treated with reduced intensity conditioning regimens, consisting of low dose total body irradiation (TBI) and Fludarabin (FAra) in the majority of the protocols, 10 of these were treated according to the FLAMSA-Protocol (Schmid C. et al, Dose-reduced conditioning before allogenic stem cell transplantation: principals, clinical protocols and preliminary results; 2002; Dtsch. Med. Wochenschr. 127:2186-2192). Sixteen patients were treated with standard intensity protocols: 8 received total body irradiation (TBI, 6×2 Gy over 3 days) and cyclophosphamide (60 mg/kg×2 days), while 8 received busulfan (4 mg/kg for 4 days) followed by cyclophosphamide (120 mg/kg for additional 2 days). Five patients received additional radioimmunotherapy (RIT).

Twenty patients were transplanted from unrelated donors (19 from matched unrelated donors (MUD), 1 mismatch), 12 patients received stem cells from HLA-identical family donors, 3 received stem cells from haploidentical family donors. Stem cell source were peripheral blood stem cells in 33 patients and bone marrow in 4 patients. Two patients with haploidentical donors received bone marrow plus peripherical blld stem cells.

GvHD prophylaxis was methotrexate (MTX) or mycophenolate mofetil (MMF) and cyclosporin A (CSA) in 32 patients and T-cell depletion in 3.

Five patients (1 MM, 1 AML, 3 NHL) were transplanted with autologous peripheral stem cells and served as controls for the allo-response and GvHD patterns in this setting. The clinical data of the patients after HSCT are summarized in Table 4.

In addition, samples were obtained from 5 patients with severe septic complications from the intensive care unit (ICU). Patterns of these patients and from 1 patient after HSCT developing sepsis were compared to the polypeptide patterns “significant” for GvHD.

Sample Preparation for Capillary Electrophoresis:

Spot urine samples were collected twice a week starting before conditioning until discharge from the ward and stored at −20° C. until analysis. Aliquots of 2 ml were adjusted to pH 10.0 using ammonia and cleared by centrifugation for 10 min at 13000×g. 2 ml were applied onto a Pharmacia C2-column to enrich for proteins and peptides and to remove urea, salts and other confounding material. Polypeptides were eluted with 50% acetonitrile in H₂O containing 0.5% formic acid, frozen and lyophilized overnight in a Christ Speed-Vac RVC₂₋₁₈/Alpha 1-2 (Christ, Osterode, Germany). The samples were resuspended in 20 μl HPLC-grade water, sonicated for 1 min and centrifuged for 10 min (13000×g at 4° C.). 100 nl were injected into the CE, while the remaining material was stored at −80° C. for further evaluation like repeating runs or sequencing. All chemicals were purchased from Merck KGaA, Germany.

Capillary Electrophoresis and Mass Spectrometry

About 100 nl of the prepared sample were injected into the CE, a P/ACE MDQ (Beckman Coulter, Fullerton, USA) system and separation was performed with +30 kV on the injection site. Upon application of high voltage the ions (polypeptides) in the sample were initially focused and subsequently separated by electrophoresis. For detection and characterization of the polypeptides, the CE was coupled on-line with an electrospray ionization time-of-flight mass spectrometer (ESI-TOF-MS). CE-ESI-MS coupling was accomplished using a CE-ESI-MS sprayer kit (Agilent Technologies, Palo Alto, USA). On-line TOF detection and data acquisition was performed on a Mariner Biospectrometry Workstation (Applied Biosystems, Farmington, USA). The data acquisition and the MS-run were automatically controlled by the CE-program via contact-close-relays. To achieve highest signal intensities, the sheath flow rate was set to a minimum (100˜1000 nl/min), while the nebulizer gas was turned off during acquisition. Under these conditions, 50 fmol of a set of different standard proteins and peptides resulted in signals with signal/noise ratios between 50 and 500. The used TOF-MS delivers the data with mass accuracy better than 100 ppm under the conditions applied. This setup enables the analysis of less than pg-amounts of polypeptides and can potentially yield the display of thousands of different polypeptides present in one individual sample without the need of any specific reagents.

Data Processing

The enormous amount of information obtained in one single CE/MS run required the development of specialized software to evaluate the data in a reproducible and automated fashion. The used software (MosaiquesVisu, biomosaiques software GmbH, Germany) recognizes MS peaks, determines the charge of each signal based on isotopic distribution and conjugated mass and subsequently generates a list of polypeptides defined by mass and migration time, which is the basis for comparison with other samples and is stored for each individual sample in the database. The signal intensity of the individual molecules is shown in a color code (ranging from 0 to 25000 MS counts) and serves as a measure for the relative abundance of particular peptides. To account for run to run variations, the CE-migration times were normalized, using 104 polypeptides present with high probability in urine samples (table 3). This allowed comparison and search of conformity within different individual samples. The signal intensity was normalized to the total ion current. Polypeptides were considered identical, if the mass deviation was less than 300 ppm and the CE migration-time deviation was less than 5 min.

Statistical Analysis:

For discrimination between healthy subjects and different groups of patients with renal diseases we used the method of Random Forests and the corresponding S-Plus program version 6/2002 Breiman L: Random Forests. (http://oz.berkeley.edu/users/breiman/randomforest2001.pdf). In this procedure, a series of PP subsets of fixed size is selected randomly from all candidate PP. For each subset, a classification tree as described in the Classification and Regression Tree (CART) analysis is generated (Steinberg D, Colla P: CART—Classification and Regression trees. San Diego, Calif., Salford Systems 1997), resulting in a classification rule. The forest prediction is the unweight plurality of class votes of the series of classification rules. Over-fitting is not generated due to large numbers of subset selections. The estimated generalisation error is unbiased due to the method of “out of bag” (oob) estimation: each tree is grown on a bootstrap sample of cases of the learning sample and the validation is estimated on the basis of those cases not selected in the bootstrap sample.

Further, discrimination between groups was also performed using support vector machines. This tool has the advantage of discriminating data in high dimensional parameter space. Its fast and stable algorithms showed good performance in the evaluation of clinical markers (Dieterle F, Muller-Hagedorn S, Liebich H M, Gauglitz G: Urinary nucleosides as potential tumor markers evaluated by learning vector quantization. Artif Intell Med 28:265-279, 2003) and different areas of biological analyses like DNA arrays (Brown M P, Grundy W N, Lin D. et al: Knowledge-based analysis of microarray gene expression data by using support vector machines. Proc Natl Acad Sci USA 97:262-267, 2000).

In a preferred embodiment, the markers are selected from markers 30 to 69 of table 1.

In a further preferred embodiment, several markers are analyzed. The preferred number of markers is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56.

TABLE 1
Discriminating polypeptide patterns for GvHD in patients after HSCT

| Polypeptide Identification | | | Marker | Frequency in group | | Mean amplitude | |
|---|---|---|---|---|---|---|---|
| No. | Mass (Da) | time (min) | Type | GvHD | control | GvHD | control |
| 1 | 1965.80 | 34.7 | GvHD | 82% | 18% | 801 | 89 |
| 2 | 1752.60 | 36.0 | GvHD | 55% | 0% | 280 | 0 |
| 3 | 1851.20 | 34.0 | GvHD | 64% | 9% | 314 | 28 |
| 4 | 1068.60 | 37.8 | GvHD | 50% | 0% | 434 | 0 |
| 5 | 1572.40 | 36.0 | GvHD | 59% | 9% | 246 | 25 |
| 6 | 1869.70 | 46.3 | GvHD | 50% | 0% | 108 | 0 |
| 7 | 3996.80 | 30.2 | GvHD | 50% | 0% | 842 | 0 |
| 8 | 1829.30 | 33.2 | GvHD | 95% | 73% | 16298 | 2047 |
| 9 | 2378.20 | 34.6 | GvHD | 18% | 73% | 53 | 301 |
| 10 | 3442.10 | 44.1 | GvHD | 45% | 100% | 4581 | 12182 |
| 11 | 1352.10 | 48.1 | GvHD | 23% | 82% | 173 | 1572 |
| 12 | 3209.50 | 33.6 | GvHD | 23% | 82% | 825 | 3605 |
| 13 | 6186.90 | 38.8 | GvHD | 23% | 82% | 275 | 2161 |
| 14 | 1731.90 | 50.1 | GvHD | 32% | 91% | 344 | 799 |
| 15 | 3092.80 | 45.9 | GvHD | 27% | 91% | 283 | 974 |
| 16 | 1848.10 | 56.6 | GvHD | 9% | 82% | 5 | 650 |
| 30 | 1082.53 | 20.86 | GvHD | 0.37 | 86.05 | 0.79 | 149.66 |
| 31 | 1085.48 | 20.63 | GvHD | 0.14 | 157.26 | 0.46 | 495.96 |
| 32 | 1098.53 | 21.27 | GvHD | 0.12 | 62.96 | 0.51 | 110.27 |
| 33 | 1127.31 | 35.18 | GvHD | 0.06 | 42.07 | 0.38 | 331.61 |
| 34 | 1155.53 | 20.82 | GvHD | 0.15 | 95.74 | 0.58 | 136.09 |
| 35 | 1196.57 | 20.89 | GvHD | 0.34 | 94.73 | 0.73 | 185.24 |
| 36 | 1226.57 | 20.99 | GvHD | 0.35 | 109.66 | 0.74 | 231.51 |
| 37 | 1250.25 | 35.80 | GvHD | 0.22 | 177.56 | 0.39 | 587.55 |
| 38 | 1290.40 | 31.36 | GvHD | 0.15 | 162.65 | 0.50 | 303.41 |
| 39 | 1392.68 | 21.83 | GvHD | 0.91 | 422.24 | 0.97 | 1329.89 |
| 40 | 1524.73 | 20.08 | GvHD | 0.28 | 93.28 | 0.64 | 207.74 |
| 41 | 1562.76 | 22.36 | GvHD | 0.38 | 156.75 | 0.82 | 384.38 |
| 42 | 1579.75 | 20.03 | GvHD | 1.00 | 747.92 | 1.00 | 2458.50 |
| 43 | 1595.76 | 20.12 | GvHD | 0.25 | 63.70 | 0.62 | 121.62 |
| 44 | 1619.81 | 40.13 | GvHD | 0.42 | 310.83 | 0.28 | 2878.45 |
| 45 | 1716.57 | 20.71 | GvHD | 0.58 | 1373.59 | 0.58 | 406.98 |
| 46 | 1829.07 | 21.16 | GvHD | 0.52 | 2406.67 | 0.43 | 583.82 |
| 47 | 1878.91 | 20.54 | GvHD | 0.62 | 1427.89 | 0.73 | 367.86 |
| 48 | 2328.04 | 21.44 | GvHD | 0.23 | 612.79 | 0.39 | 202.60 |
| 49 | 2404.10 | 20.15 | GvHD | 0.35 | 111.56 | 0.76 | 221.05 |
| 50 | 2427.27 | 19.61 | GvHD | 0.38 | 888.77 | 0.14 | 268.64 |
| 51 | 2540.48 | 19.66 | GvHD | 0.37 | 2470.99 | 0.22 | 683.65 |
| 52 | 2570.27 | 42.58 | GvHD | 0.46 | 229.21 | 0.58 | 746.97 |
| 53 | 2603.39 | 20.01 | GvHD | 0.46 | 6143.60 | 0.27 | 1688.52 |
| 54 | 2682.24 | 22.19 | GvHD | 0.23 | 121.09 | 0.59 | 138.59 |
| 55 | 2708.42 | 23.09 | GvHD | 0.75 | 491.90 | 0.38 | 458.20 |
| 56 | 2740.58 | 23.17 | GvHD | 0.74 | 571.77 | 0.39 | 542.56 |
| 57 | 2898.10 | 28.74 | GvHD | 0.35 | 5342.04 | 0.55 | 677.80 |
| 58 | 2923.48 | 20.25 | GvHD | 0.72 | 2488.14 | 0.36 | 2136.17 |
| 59 | 3138.99 | 30.35 | GvHD | 0.48 | 198.41 | 0.66 | 597.93 |
| 60 | 3143.56 | 33.74 | GvHD | 0.28 | 322.03 | 0.63 | 858.37 |
| 61 | 3158.52 | 28.84 | GvHD | 0.37 | 251.09 | 0.72 | 414.33 |
| 62 | 3205.51 | 19.83 | GvHD | 0.35 | 42.95 | 0.63 | 149.30 |
| 63 | 3303.49 | 30.34 | GvHD | 0.25 | 250.49 | 0.65 | 275.70 |
| 64 | 3310.52 | 24.71 | GvHD | 0.63 | 371.30 | 0.25 | 336.10 |
| 65 | 3530.74 | 25.68 | GvHD | 0.03 | 167.44 | 0.38 | 296.34 |
| 66 | 4306.05 | 24.59 | GvHD | 0.86 | 1435.22 | 0.46 | 1189.74 |
| 67 | 4863.22 | 26.19 | GvHD | 0.37 | 305.37 | 0.74 | 485.38 |
| 68 | 8559.58 | 19.67 | GvHD | 0.65 | 917.11 | 0.30 | 730.24 |
| 69 | 9866.82 | 20.87 | GvHD | 0.18 | 101.88 | 0.65 | 290.86 |

TABLE 2
Discriminating polypeptide patterns for Sepsis in patients after HSCT

| Polypeptide Identification | | | Marker | Frequency in group | | | mean amplitude | | |
|---|---|---|---|---|---|---|---|---|---|
| No | mass (Da) | time (min) | Type | GvHD | control | Sepsis | GvHD | control | Sepsis |
| 17 | 1542.90 | 50.7 | Sepsis | 18% | 9% | 83% | 33 | 7 | 267 |
| 18 | 2165.00 | 42.1 | Sepsis | 18% | 9% | 83% | 114 | 97 | 604 |
| 19 | 1238.80 | 32.4 | Sepsis | 23% | 27% | 92% | 141 | 227 | 549 |
| 20 | 2052.80 | 46.2 | Sepsis | 18% | 18% | 83% | 38 | 46 | 169 |
| 21 | 2145.80 | 41.6 | Sepsis | 36% | 36% | 100% | 877 | 290 | 1948 |
| 22 | 1104.60 | 46.0 | Sepsis | 23% | 18% | 83% | 108 | 102 | 330 |
| 23 | 1602.80 | 39.6 | Sepsis | 27% | 9% | 83% | 304 | 28 | 534 |
| 24 | 1809.90 | 40.6 | Sepsis | 32% | 55% | 100% | 255 | 217 | 878 |
| 25 | 1854.60 | 51.6 | Sepsis | 27% | 36% | 92% | 87 | 464 | 306 |
| 26 | 3002.00 | 47.2 | Sepsis | 59% | 64% | 0% | 240 | 877 | 0 |
| 27 | 3385.50 | 37.0 | Sepsis | 55% | 73% | 0% | 775 | 1348 | 0 |
| 28 | 3840.60 | 25.8 | Sepsis | 59% | 73% | 0% | 3635 | 2219 | 0 |
| 29 | 4044.70 | 29.2 | Sepsis | 59% | 73% | 0% | 1751 | 1405 | 0 |

TABLE 3
Internal standards to standardize the CE-time

| migration time [min] | dt [min] | mass [Da] |
|---|---|---|
| 15.490396 | 0.158804 | 8054.473633 |
| 15.803237 | 0.155143 | 8765.233398 |
| 16.034266 | 0.174906 | 1621.9104 |
| 16.185061 | 0.147871 | 9180.99707 |
| 16.645294 | 0.198704 | 10045.20703 |
| 17.663696 | 0.165531 | 10388.81348 |
| 17.980883 | 0.178564 | 10518.18457 |
| 19.917442 | 0.234131 | 9220.939453 |
| 20.34516 | 0.170572 | 1877.789429 |
| 20.479975 | 0.221246 | 3842.693604 |
| 20.519386 | 0.265078 | 4747.932617 |
| 21.804012 | 0.271715 | 4240.856445 |
| 22.221563 | 0.191069 | 4282.796387 |
| 22.777784 | 0.245503 | 3840.540527 |
| 24.304148 | 0.319715 | 7556.177734 |
| 24.579231 | 0.291986 | 879.519653 |
| 24.813087 | 0.224198 | 1867.731689 |
| 25.283239 | 0.22054 | 2266.040771 |
| 26.177101 | 0.289898 | 2172.188721 |
| 26.773794 | 0.352887 | 2914.05542 |
| 26.81407 | 0.297343 | 962.591919 |
| 28.254925 | 0.581783 | 4353.585938 |
| 29.822325 | 0.595913 | 1682.720947 |
| 30.75272 | 0.175961 | 943.492859 |
| 30.762201 | 0.263861 | 1108.647949 |
| 30.926645 | 0.138075 | 1368.781738 |
| 31.305229 | 0.301605 | 3987.548828 |
| 31.433071 | 0.515308 | 1099.419434 |
| 32.165497 | 0.198377 | 3122.730713 |
| 32.222111 | 0.226858 | 1829.089966 |
| 33.427856 | 0.151562 | 2767.015625 |
| 34.053886 | 0.252424 | 1302.691772 |
| 34.681156 | 0.20976 | 3685.918213 |
| 35.30254 | 0.207782 | 2389.097168 |
| 35.502213 | 0.388916 | 3209.800293 |
| 36.314056 | 0.183495 | 980.526123 |
| 36.404907 | 0.145751 | 1008.513733 |
| 36.424831 | 0.150486 | 1000.48761 |
| 36.720509 | 0.128397 | 2717.472656 |
| 36.777012 | 0.164648 | 2663.246826 |
| 37.557594 | 0.165628 | 3556.580566 |
| 37.572525 | 0.185484 | 1743.890381 |
| 37.680653 | 0.160958 | 1134.580566 |
| 37.700241 | 0.171622 | 4097.981934 |
| 38.050472 | 0.156383 | 3152.361572 |
| 38.155159 | 0.217341 | 2825.309082 |
| 38.17057 | 0.432096 | 882.532654 |
| 38.281631 | 0.20781 | 996.190369 |
| 38.57658 | 0.370648 | 1425.324829 |
| 38.687305 | 0.15052 | 3385.513916 |
| 38.830559 | 0.056085 | 1352.824097 |
| 38.921108 | 0.150325 | 5000.982422 |
| 39.241917 | 0.178206 | 3775.720459 |
| 39.433277 | 0.235333 | 3405.60791 |
| 39.484215 | 0.140887 | 1046.52771 |
| 39.513248 | 0.093703 | 2154.053955 |
| 39.936756 | 0.195951 | 6171.129395 |
| 40.533363 | 0.158628 | 1194.581543 |
| 40.686531 | 0.122381 | 1265.634888 |
| 40.83009 | 0.191972 | 2642.264893 |
| 41.506096 | 0.161887 | 4159.304199 |
| 41.818069 | 0.163642 | 2742.253418 |
| 42.079609 | 0.266392 | 1463.643311 |
| 42.636433 | 0.041732 | 1487.660034 |
| 42.811199 | 0.246696 | 1579.670776 |
| 42.940624 | 0.1884 | 3121.243164 |
| 43.093792 | 0.106392 | 3271.523438 |
| 43.115334 | 0.607341 | 1834.878052 |
| 43.46143 | 0.193155 | 3442.135498 |
| 43.494144 | 0.20218 | 3495.841797 |
| 43.549488 | 0.217899 | 3473.905029 |
| 43.740391 | 0.12795 | 3108.919434 |
| 44.191006 | 0.18629 | 3359.583496 |
| 44.230297 | 0.233319 | 3416.526611 |
| 44.934914 | 0.127421 | 1991.917114 |
| 45.538418 | 0.214716 | 2197.337158 |
| 45.675098 | 0.12333 | 1889.864502 |
| 46.313114 | 0.259721 | 2385.597168 |
| 47.216648 | 0.168651 | 2649.602539 |
| 47.279705 | 0.127824 | 2343.072998 |
| 47.526871 | 0.19233 | 2584.635986 |
| 48.441795 | 0.239347 | 1160.526001 |
| 48.804813 | 0.251244 | 1261.53125 |
| 49.519478 | 0.243133 | 1274.625244 |
| 51.492035 | 0.213235 | 1211.559204 |
| 51.657627 | 0.822884 | 1223.348633 |
| 53.168346 | 0.293424 | 1351.643433 |
| 53.240913 | 0.216809 | 1367.655151 |
| 53.259499 | 0.15916 | 1770.30481 |
| 54.59832 | 0.234281 | 1507.742432 |
| 55.038143 | 0.329349 | 1594.211426 |
| 57.475471 | 0.325805 | 1840.810547 |
| 58.898354 | 0.484288 | 2608.239746 |
| 60.082333 | 0.507699 | 1863.939453 |

1. A method for the diagnosis of GvHD, the method comprising:
a) measuring the presence or the absence of a polypeptide marker in a urine sample, wherein the polypeptide marker is selected from the group of polypeptide markers shown in table 1, and
b) comparing the probability of the presence of this marker in a disease patient to the probability of the presence of this marker in a control patient, wherein
c1) if the probability of the presence of this marker in a disease patient is higher than the probability of the presence of this marker in a control patient, the presence of this marker is indicative for a higher probability of having the disease rather than the control condition, or
c2) if the probability of the presence of this marker in a disease patient is lower than the probability of the presence of this marker in a control patient, the absence of the marker is indicative for a higher probability of having the disease rather than the control condition.

2. The method according to claim 1, wherein the individual probabilities in step b) are as indicated in the table 1,

3. The method according to claim 1, wherein the control represents a healthy condition.

4. The method according to claim 1, wherein the method comprises detecting a plurality of the polypeptide markers selected from table 1.

5. The method according to claim 1, wherein the method comprises detecting at least 3 or at least 10 polypeptide marker.

6. The method of claim 5, wherein the peptide markers are selected from polypeptide marker No. 1, 2 and 3; 2, 3 and 4; 3, 4 and 5; 16, 15 and 14; 15, 14 and 13; 14, 13 and 12; or 1, 2 and 16; 2, 3 and 16; 3, 4 and 16; 1, 2 and 15; 2, 3 and 15; 3, 4 and 15; 1, 2 and 14; 2, 3 and 14; 3, 4, and 14.

7. The method of claim 5, wherein the marker are selected from polypeptide marker No. 1-10; 7-16; or 1-5 and 12 16.

8. The method according to claim 1, wherein the method comprises detecting all of the polypeptide markers from table 1.

9. A method for the differential diagnosis between GvHD and Sepsis, the method comprising:
a) measuring the presence or the absence of a polypeptide marker in a urine sample, wherein the polypeptide marker is selected from the group of polypeptide markers shown in table 2, and
b) comparing the probability of the presence of this marker in a GvHD patient to the probability of the presence of this marker in a Sepsis patient, wherein
c1) if the probability of the presence of this marker in a GvHD patient is higher than the probability of the presence of this marker in a Sepsis patient, the presence of this marker is indicative for a higher probability of having GvHD rather than Sepsis, or
c2) if the probability of the presence of this marker in a GvHD patient is lower than the probability of the presence of this marker in a Sepsis patient, the absence of the marker is indicative for a higher probability of having GvHD rather than Sepsis.

10. The method according to claim 9, wherein the individual probabilities in step b) are as indicated in the table 2.

11. The method according to claims 9, wherein the method comprises detecting a plurality of the polypeptide markers selected from table 2.

12. The method according to claim 11, wherein the method comprises detecting at least 3 of the polypeptide markers selected from table 2.

13. The method of claims 12, wherein the polypeptide markers are selected from polypeptide marker No. 17, 18 and 19; 18, 19 and 20; 19, 20 and 21; 20, 21 and 22; or 29, 28 and 27; 28, 27 and 26; or 17, 18 and 29; 18, 19 and 29; 19, 20 and 29; or 17, 18 and 28; 18, 19 and 28; 19, 20 and 28.

14. The method according to claim 11, wherein the method comprises detecting at least 10 of the polypeptide markers selected from table 2.

15. The method according to claim 14, wherein the polypeptide marker is selected from polypeptide marker No. 17-26; 20-29; or 17-21 and 25-29.

16. The method according to claim 9, wherein the method comprises detecting all of the polypeptide markers.

17. The method according to claim 1, wherein ELISA, quantitative Western Blot, radio-immuno-assay, surface plasmon resonance, array, gel electrophoresis, capillary electrophoresis, gas phase ion spectrometry, or mass spectrometry is used for detecting the presence of the marker or markers.

18. The method according to claim 1, wherein the polypeptide markers in the sample are separated by capillary electrophoresis before measurement.

19. The method according to claim 18, wherein mass spectrometry is used for detecting the presence of the marker or markers.
